(19) United States
(12) Patent Application Publication
(10) Pub. No.: US 2026/0041468 A1
(43) Pub. Date: Feb. 12, 2026

(54) KYPHOPLASTY ONE PASS CANNULA DEVICES, SYSTEMS AND METHODS

(71) Applicant: Amazing Brace LLC, Tampa, FL (US)

(72) Inventor: Victor Hayes, Tampa, FL (US)

(21) Appl. No.: 19/294,432

(22) Filed: Aug. 8, 2025

Related U.S. Application Data
(60) Provisional application No. 63/681,072, filed on Aug. 8, 2024.

(51) Int. Cl. A61B 17/88; A61B 10/02

(52) U.S. Cl. CPC A61B 17/8816; A61B 10/025; A61B 17/8811

(57) ABSTRACT

Provided are kyphoplasty cannula devices and device systems that include an outer sheath including a main body defining a through-opening extending therethrough, and an outer sheath handle above the main body, the outer sheath handle having a handle opening through at least one end thereof, the outer sheath handle also having a top opening that is contiguous with the through-opening of the main body; and an inner stylet configured to be insertable into the top opening of the outer sheath handle and into the through-opening of the main body. The present systems may also include a plug to plug the top opening after removal of the inner stylet. Also provided are kits and methods of using the present devices and sytems.

### CROSS-REFERENCE TO RELATED APPLICATION

This application claims priority to and benefit of U.S. Provisional Application 63/681,072 filed on Aug. 8, 2024, the entire contents of which are hereby incorporated by reference.

### FIELD

The present application relates to cannula instruments/devices and systems for use in Kyphoplasty procedures, for example, under local anesthetic, and methods of assembling and using the present instruments/devices and systems to perform a Kyphoplasty procedure on a patient in need of a Kyphoplasty procedure. The present instruments, systems and methods are advantageous at least because they reduce the number of instruments passed within and through the pedicle of a patient during a Kyphoplasty procedure.

### BACKGROUND

Compression fractures within the thoracic and lumbar spine are common entities encountered in medicine. As the human population continues to age, osteoporosis becomes more prevalent, and the number of compression fractures has steadily increased over time. Therefore, the number of Kyphoplasty procedures is increasing to restore damaged vertebra height after a compression fracture and relieve pain in the patient. It may be generally preferred to perform these procedures under local anesthetic, when they can be safely performed on the local anesthetic, avoiding the risk of general anesthesia.

Performing these procedures within a patient under purely local anesthetic and on an awake patient can have its difficulties, however. The systems that are currently in use for Kyphoplasty procedures are standard Jamshidi™ needles, along with drills, tamps, and additional biopsy instruments. Cement is typically pushed through preloaded tubes. Using the standard equipment above requires passing multiple instruments through the initial Jamshidi™ cannula. With an awake patient under local anesthetic, the more times the instruments are passed in and out of the pedicle system the more chance there is of pedicle perforation and increased pain.

### BRIEF SUMMARY

Provided herein are novel one pass or one entry cannulated instruments/devices that can be screwed into the pedicle of a patient's vertebrae. The present devices may be used for procedures that include Kyphoplasty alone or for procedures that also include biopsies. The present application advantageously removes the need for separate drills and biopsy equipment and reduces the number of instruments passed within and through the pedicle. The present devices also avoid hammering the Jamshidi™ into the pedicle, which can be painful, particularly in an awake patient.

The present systems include an outer sheath including a main body defining a through-opening extending therethrough, and an outer sheath handle above the main body, the outer sheath handle having a handle opening through at least one end thereof, the outer sheath handle also having a top opening that is contiguous with the through-opening of the main body; and an inner stylet configured to be insertable into the top opening of the outer sheath handle and into the through-opening of the main body. The present systems may also include a plug to plug the top opening after removal of the inner stylet.

Also provided are kits that include one or more of the present instruments/devices and at least one additional component as part of the system or to assist in use or assembly of the present systems.

Further provided are methods for assembling the present devices and for performing a Kyphoplasty procedure with and without biopsies, utilizing the present instruments and systems.

### BRIEF DESCRIPTION OF THE FIGURES

Non-limiting embodiments of the present application are demonstrated with reference to the attached figures.

FIGS. 1-2 depict an outer sheath of a cannula instrument (or “device”) in accordance with non-limiting example embodiments. The outer sheath includes a handle as a top portion of the outer sheath.

FIGS. 3-5 depict three different inner stylet options for the cannula instrument, each of which are within the scope of non-limiting embodiments. The inner stylet is configured to pass into and via the handle of the outer sheath, and may be locked into the outer sheath, in accordance with non-limiting example embodiments.

FIG. 6 depicts an example of a non-limiting inner stylet option and outer sheath assembled into a cannula instrument ready for use in accordance with example embodiments. Other inner stylet options, such as the other options depicted in FIGS. 3-5, are contemplated and within the scope of the present application.

FIG. 7 depicts an example locking mechanism that is configured to secure the stylet handle to the outer sheath handle in accordance with non-limiting example embodiments.

FIGS. 8-9 depict a handle opening on the outer sheath with an instrument that may be inserted into the handle, so the outer sheath can be twisted over the inner stylet. The screw threads on the inside of the outer sheath or the outside of the inner stylet, that are mated with one another, optionally, just below the handle will allow the outer sheath to be screwed into the bone and pushed down.

FIG. 10 depicts the action of the screw-in of the outer sheath over the stylet. After the outer sheath is lowered and covers the threads of the stylet, then the stylet is removable from the outer sheath containing any biopsies of the patient, which are caught between the threads as it is being screwed and unscrewed and removed from the patient. In embodiments in which the inner stylet is configured to remove a core biopsy, the stylet is removable from the outer sheath containing a core biopsy from the patient.

FIG. 11 depicts a cannula instrument or device in accordance with non-limiting example embodiments, for example, after a stylet has been removed from the outer sheath. The outer sheath may be configured for cement injection, for example of bone cement, directly through the handle.

FIG. 12 depicts a non-limiting example embodiment of a plug that may be included as part of the present systems, to allow cement injection. The plug of FIG. 12 may be configured e.g., for cement injection directly through the handle. The plug may be configured to be screwed into threads of the outer sheath, after removal of the inner stylet.

FIG. 13 depicts a non-limiting embodiment showing a plug to the left of a cannula and within a cannula instrument or device of the present systems that allow for cement injection directly through the handle of the instrument or device.

FIG. 14 depicts a non-limiting embodiment showing an example plug configured for insertion within a cannula instrument or device of the present systems, which plug is configured for cement injection using small pushers and prefilled cement cannulas.

FIG. 15 depicts a non-limiting embodiment showing a plug within a cannula instrument or device of the present systems that allows for cement injection using small pushers and prefilled cement cannulas. FIG. 15 depicts the plug inserted into the instrument or device.

### DETAILED DESCRIPTION

A goal of the present devices and methods is to create a one-step Kyphoplasty system where once the needle is passed into the pedicle of a patient, a biopsy can be taken and the cement can be directly injected into the desired location of the patent, with minimal nipple lesion. The present devices and systems may be of particularly useful or of the utmost importance when a practitioner is performing these procedures under local anesthetic, at least to reduce the chance of pedicle performation and to avoid increased pain.

Accordingly, the present application relates to cannula instruments/devices and systems for use in Kyphoplasty procedures, for example, under local anesthetic, and methods of assembling and using the present instruments/devices and systems to perform a Kyphoplasty procedure, which may reduce the number of instruments passed within and through the pedicle during a procedure. Also provided are kits that include on or more of the presently disclosed instruments or devices or components thereof. The present devices and systems are specifically designed to be used under local anesthetic to perform kyphoplasty's with and without biopsies. It is contemplated however, that they may be used under general anesthesia, if need be.

While embodiments are described herein, it is to be understood that this disclosure is illustrative and exemplary and is made for the purposes of providing a full and enabling disclosure. The detailed disclosure herein of example embodiments is not intended, nor is to be construed, to be limiting. All illustrations of the figures are for the purpose of describing selected embodiments and are not intended to limit the scope of the present application.

As should be understood, any embodiment may incorporate only one or a plurality of the above-disclosed aspects of the disclosure and may further incorporate only one or a plurality of the above-disclosed features. Items described in the singular herein may be provided in plural, as can be seen, for example, in the drawings. Thus, the description of a single item that is provided in plural should be understood to be applicable to the remaining plurality of items unless context indicates otherwise. When used herein to join a list of items, “or” denotes “at least one of the items,” but does not exclude a plurality of items of the list.

Each term used herein refers to that which an ordinary artisan would understand such term to mean based on the contextual use of such term herein. To the extent the meaning of a term used herein—as understood by the ordinary artisan differs in any way from any particular dictionary definition of such term, the meaning of the term as understood by the ordinary artisan in the context of the present application, should prevail.

The terms “a” or “an”, as used herein, are defined as one or more than one. The term “plurality,” as used herein, is defined as two or more than two. The term “another,” as used herein, is defined as at least a second or more.

Throughout the specification, when a component is described as “including” or “having” or “comprising” a particular element or group of elements, it is to be understood that the component is formed of only the element or the group of elements, or the element or group of elements may be combined with additional elements to form the component, unless the context indicates otherwise.

It will be understood that when an element is referred to as being “connected” or or “on” another element, it can be directly connected or on the other element or intervening elements may be present.

Spatially relative terms, such as “lower,” “top,” “front,” “left” and the like, may be used herein for ease of description to describe positional relationships, such as illustrated in the figures, for example. It will be understood that the spatially relative terms encompass different orientations of the device in addition to the orientation depicted in the figures.

According to non-limiting example embodiments, the present cannula instruments (also referred to herein interchangably as “devices”) include at least an outer sheath and an inner stylet.

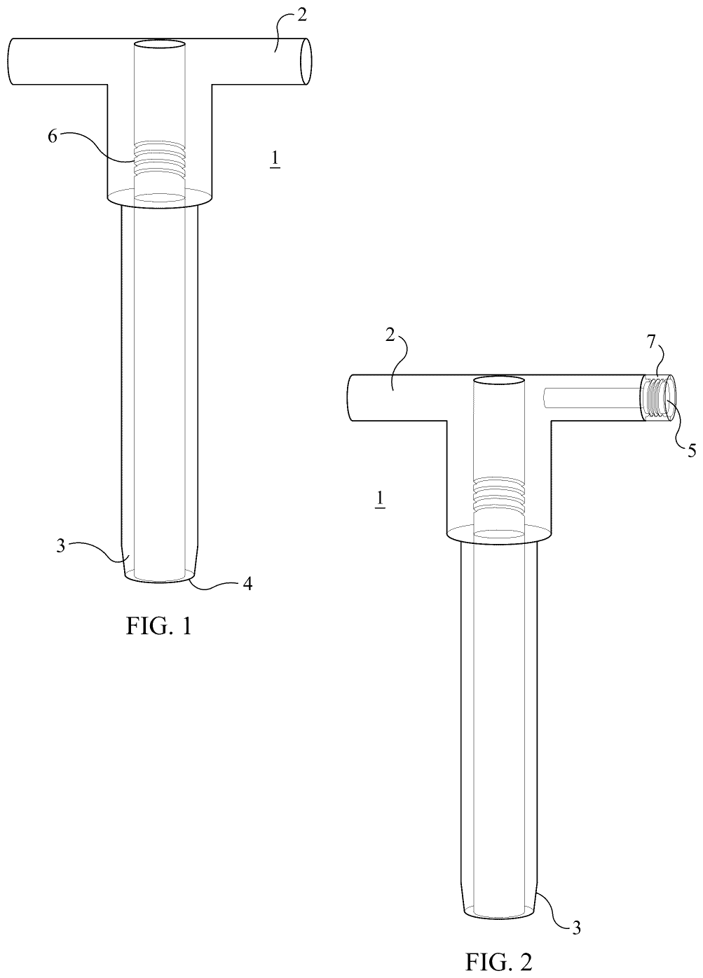

FIGS. 1-2 depict non-limiting example embodiments of outer sheaths (1) of a cannula instrument or device in accordance with the present application. The outer sheath of the cannula instrument includes a handle portion (2) at the top of the outer sheath. Although the handle, or handle portion, depicted in the present figures is T-shaped, other embodiments are envisioned that may have different lengths or circumferences on each side, may have three or more spokes (rather than the two depicted spokes), may have curvatures, etc. (for example for easier gripping or ergonomics or for increased torque or leverage, etc.), as long as the handle is configured to be able to accomplish the methods herein. It is also envisioned that the entire outer sheath may be created as one piece, or the handle portion may be created separately from a lower sheath portion and attached thereto.

The materials of various components may also be the same or different from one another. The handle and/or other portion(s) of the outer sheath may have a coating. A coating of the handle may be a different coating from a coating of (or non-coating) of the portion of the outer sheath that is inserted into a patient, for example a main body of the outer sheath. For example, it may be desirable that the sheath portion to be inserted into a patient (such as a lower sheath portion or main body) may comprise (or be coated with) a smooth material for ease of insertion. It may be desired that the handle portion of the outer sheath comprises or is coated with a material that may provide better gripping properties for a surgeon using the present devices. The handle and main body or lower sheath portion may also have a different thickness of material from one another. It is also contemplated that a lower portion (3) of the sheath may be angled as shown in the figures, or may be straight. It is further contemplated that the outer sheath may have a different configuration with a non-circular cross-section, such as a semicircular, oblong, oval, rectangular, square, trapezoidal, cross-section etc. The width or diameter of a cross-section of the lower portion of the sheath may be the same or different from a width or diameter of a cross-section of the handle portion.

According to non-limiting example embodiments, at least a portion of the outer sheath comprises a stainless and/or titanium material, but embodiments are not limited thereto. Other materials suitable for insertion into a patient are encompassed herein. For example, according to example embodiments, components may be a rigid or semi-rigid material for stability and/or to hold their shapes.

Examples of the outer sheath may include a sharp tip (4) at an end of the sheath distal to the handle, so the sheath can cut through/into bone of the patient, and optionally cut biopsy cores when the outer sheath is screwed down or pushed down over an inner stylet. In example embodiments of the present application, there is an access port (5) as shown in FIG. 2, for example in the handle of the outer sheath, which can be used to place within the outer sheath, an instrument that may be used to screw the device down into the bone of the patient.

As depicted in FIG. 1, the outer sheath (1) may include one or more sets of screw threads (6) therein (or is configured to receive screw threads) to enable screwing of other portions of the present devices and systems therewith. For example, there may be a portion of the outer sheath that has threads therein configured to be able to screw over an inner stylet, or is configured to receive threads of the inner stylet. Another portion of the outer sheath, in the handle, may have threads (7) that are related to a locking mechanism for locking the inner stylet in the outer sheath. Alternatively, a lock may be at a more central portion of the handle, not at an end of the handle.

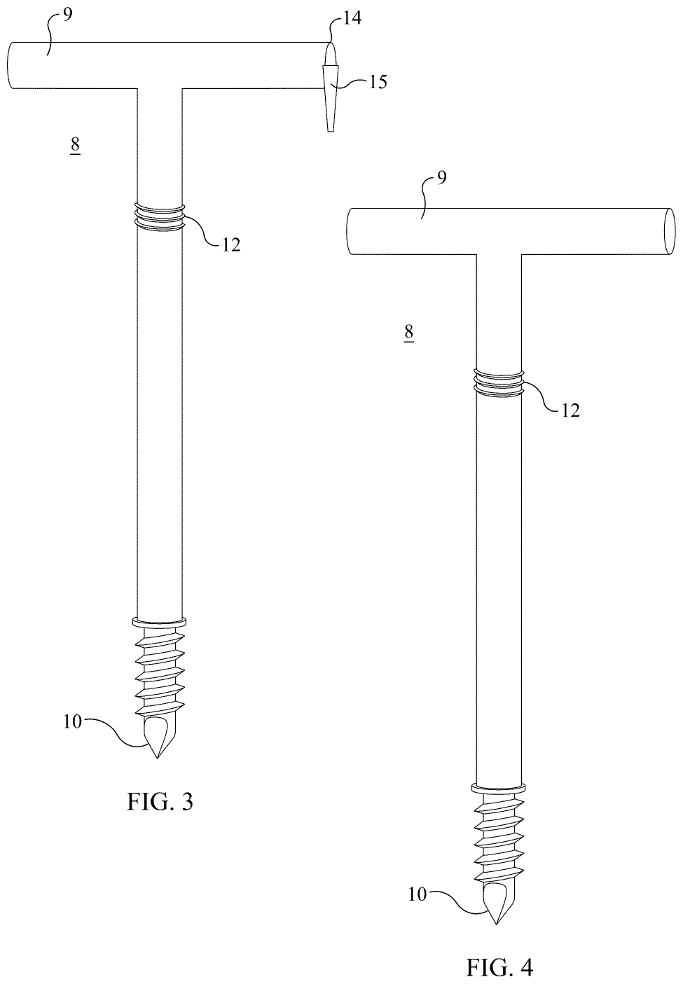

FIGS. 3-5 depict three different inner stylet (8) options for the cannula instrument according to different non-limiting embodiments, each of which are within the scope of non-limiting embodiments of the present application. The inner stylet may be locked into the outer sheath, in accordance with non-limiting example embodiments.

According to examples, the inner stylet may be configured to be passed through the cannulation of the outer sheath. The inner stylet also includes a handle portion (9). FIGS. 3-5 depict three different non-limiting embodiments of inner stylet options for the cannula instrument, each of which are within the scope of non-limiting embodiments of the present application. Each embodiment of the inner stylet is configured to pass into the outer sheath, and may be locked into the outer sheath, in accordance with non-limiting example embodiments. As depicted for example in FIG. 6, a main body portion of the inner stylet may be inserted into the outer sheath, while a handle portion of the inner stylet (9) has at least a portion that remains outside (e.g. above) the handle portion of the outer sheath (2).

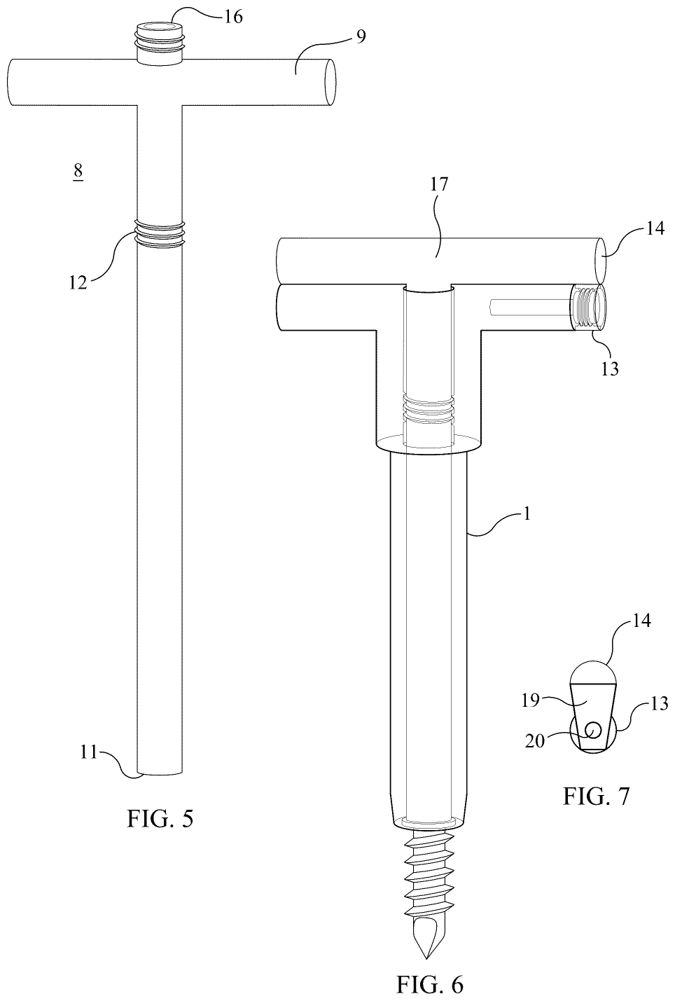

In non-limiting example embodiments, for example as shown in FIGS. 3 and 4, the inner stylet (8) includes a self drilling, self tapping tip (10). According to another non-limiting example embodiment, for example in FIG. 5, the inner stylet may include an end (11) that is configured for a core biopsy, rather than having a self drilling self tapping tip.

As shown in FIGS. 3-5, the inner stylet may have one or more threaded portion (12) thereon. At least one stylet threaded portion may be configured to pair with a the outer sheath to permit them to screw partially or fully into one another, or change height with respect to one another. The threaded portion (12) of the inner stylet (8) may be the same or different length as a corresponding threaded portion within the outer sheath (8), to allow for changing height of the inner stylet (8) within the outer sheath (1) based on how far the inner stylet is screwed into the outer sheath.

In example embodiments, the inner stylet (8) may also be locked into the outer covering sheath (1), for example using a lock or lock portion (13) of the outer sheath, for example at an end of the handle (2) of the outer sheath (1). The lock portion (13) of the outer sheath can also be used to inject cement through the present devices systems to a desired location of a patient, as discussed further herein.

The inner sylet (8) may also include one or more portions (14) configured to enable locking of the outer sheath to the inner stylet. FIG. 3 depicts, for example, a clip (15) on an end of the inner stylet handle (9) which clip (15) is configured to clip to a luer lock of the outer sheath handle. FIG. 5 depicts an example in which a luer lock (16) is at a more central location of the inner stylet/outer sheath, rather than being at an end of a handle of the outer sheath. It is contemplated that lock portions may be on either or both of the outer sheath or the inner stylet, at various locations of either or bot hof the outer sheath or the inner stylet to enable the locking of one with respet to the other.

FIG. 6 depicts a non-limiting example of an assembled device or system of the present application, which includes a possible inner stylet option (17), and an outer sheath (1) assembled into a cannula instrument. The depicted example is ready for use in accordance with example embodiments of the present application. In the depicted assemble embodiment, the inner stylet (17) has been inserted into the outer sheath (1). This depicted example shows an inner stylet (17) that has been screwed into the outer sheath (1). The inner stylet and outer sheath may be locked at the end of the handle with a lock as depicted for example, in FIG. 7. As shown in FIG. 6, a portion of the inner stylet (17) handle may remain above the handle portion of the outer sheath (2).

As indicated above, the present devices, systems, and/or kits may additionally include a locking mechanism that secures the stylet handle into the outer sheath handle. FIG. 7 depicts a front view of an example of a locking mechanism, in which a snap (19) on the inner stylet is used to hold handle portions of the outer sheath (2) and inner stylet (9) together. Shown is a non-limiting embodiment that snaps into a luer lock hole (20). The snap (19) may be on either the handle of the outer sheath or the handle of the inner stylet (as shown for example in FIG. 3). The leur lock (16) may alternatively be for example in the middle of the stylet or outer sheath as shown for example in FIG. 5.

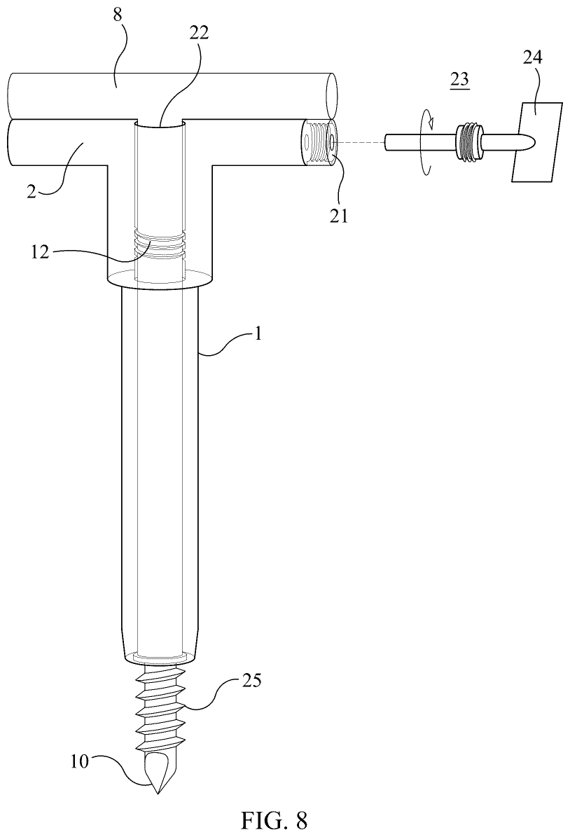

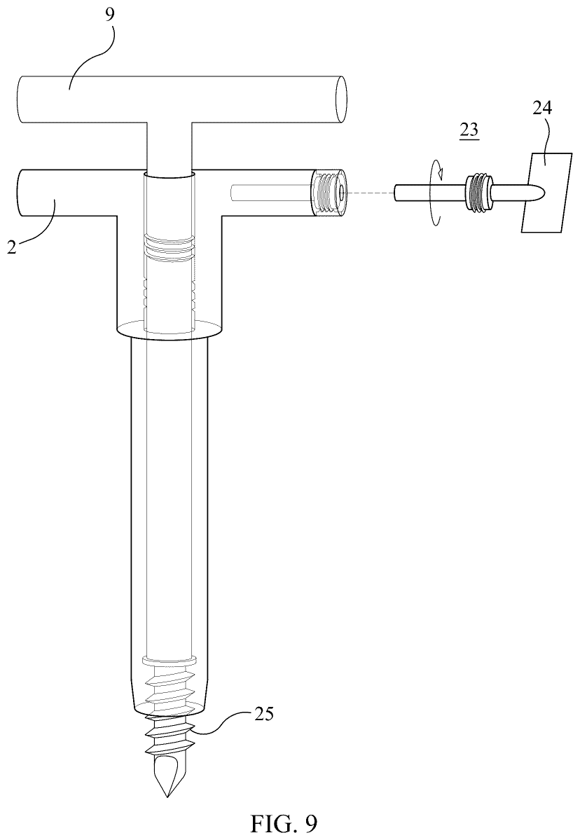

FIGS. 8-9 depict a handle opening (21) on the outer sheath (1) with an instrument (23) configured to be inserted into the handle (2), so the outer sheath (1) can be twisted over the inner stylet. The screw threads (6) on the inside of the outer sheath (1) or (18) or on the outside of the inner stylet (12), that are configured such that they may be mated with one another, optionally, just below the handle, will allow the outer sheath to be screwed into the bone of the patient and if desired, pushed down onto the bone.

FIG. 8 depicts an assembled device or system in accordance with embodiments of the present application, of an inner stylet (8) within an outer sheath (1) through opening (22), having a handle opening (21) on a distal end of a handle of the outer sheath (2). FIG. 8 shows an instrument or “key” (23) inserted into the handle opening (21), which instrument or key (23) is used to twist the outer sheath (1) over the inner stylet (8). The instrument inserted into the handle may be part of systems or kits in accordance with the present application.

The threads that are mated on the inside of the outer sheath and on the outside of the inner stylet, optionally, just below the handle will allow the outer sheath to be screwed into the bone and pushed down, for example by use of a key as shown in FIG. 8. According to example embodiments, the key may include a handle portion (24) which when turned, turns the key (23) and rotates the outer sheath (1) over the inner stylet (8) for example, in a clockwise fashion, advancing the sheath into a desired position for cement injection to the patient. According to example embodiments, a practitioner or surgeon can place a key (23), such as a small metal key in the handle opening (21) of the outer sheath handle (9) or luer lock hole (20) to start a threading process of the outer sheath being pushed toward the tip (10) of the inner stylet (8) over the stylet into the bone of the patient, covering the threaded tip as shown for example in FIG. 9. According to non-limiting examples, the key (23) may comprise a material other than metal.

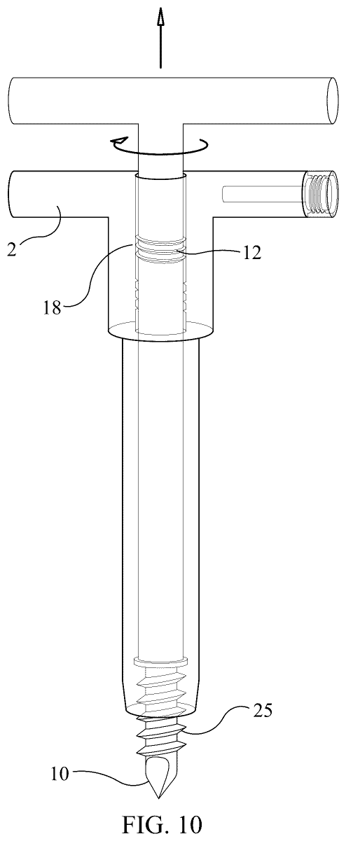

FIG. 10 depicts stages of the screw-in of the outer sheath over the stylet according to example embodiments. In example methods after the outer sheath (1) is lowered and covers the threads of the stylet (8), then the stylet is removable from the outer sheath containing any biopsies of the patient, which are caught between the threads (25) of the tip (10) as it is being screwed and unscrewed and removed from the patient. The threads (25) of the tip (10) that are initially screwed into the bone may have different patterns that may be designed to allow capture of bone shards/biopsy material while screwing the mechanism into the bone. This allows for a one step biopsy. Different thread configurations may be used.

In embodiments in which the inner stylet (8) is configured to remove a core biopsy, the stylet (such as the stylet shown in FIG. 5) is removable from the outer sheath containing a core biopsy from the patient that may be obtained due to the configuration of the end (11) of the inner stylet in the configuration of the inner stylet (8) shown in FIG. 5, which may be pushed into the patient to remove a core sample of the patient.

In particular, as shown in FIG. 9, a key (23) is inserted into the handle of the outer sheath, and rotated for advancing the sheath with respect to the inner stylet (9). Optionally a cylinder, such as a small metal cylinder, may be inserted in a hole to start the threading, which will push the outer cannula into the bone. As shown in FIG. 10, a lower handle (2) of the outer sheath rotates in over threads in the central inner stylet, creating a sheath over threads (25) of the screw tip (10). The threaded stylet may be removed from the outer sheath and any shards of bone will then be removed for a biopsy specimen.

FIGS. 11-15 depict non-limiting example embodiments of the present devices and systems and plugs that may be included as part of the present devices and systems, which may be adapted to receive the plugs, to allow for two types of cement injection: 1) directly through the handle or 2) using small pushers and prefilled cement cannulas. The plug may be configured to be screwed into threads of the outer sheath for example, after removal of the inner stylet.

FIG. 11 depicts a cannula instrument or device in accordance with non-limiting example embodiments, configured for cement injection directly through the handle (2) of the outer sheath (1) after removal of an inner stylet (8).

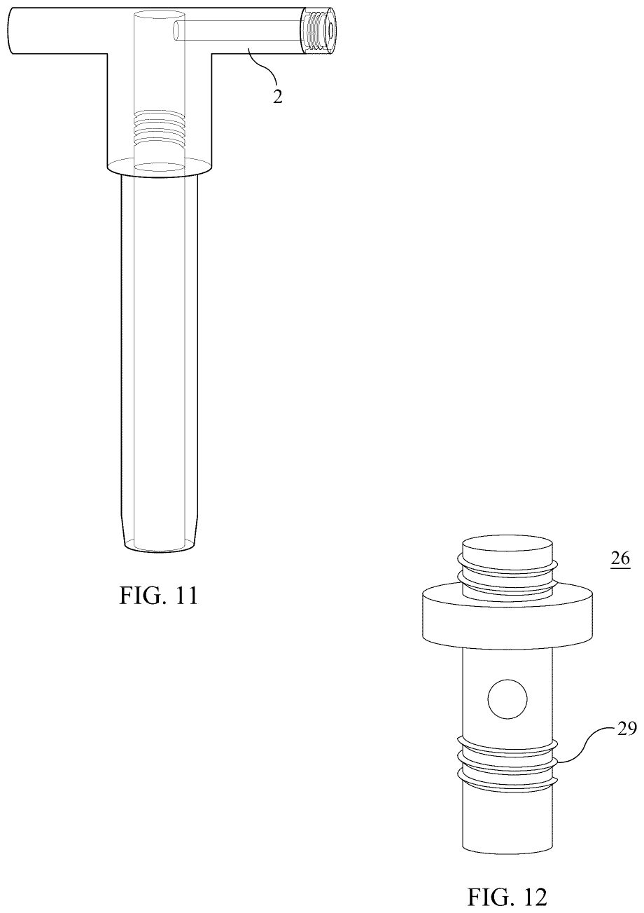

FIG. 12 depicts a non-limiting example embodiment of a plug (26) that may be included as part of the present systems, to allow for cement injection directly through the handle. The plug may be configured to be screwed into threads of the outer sheath, after removal of the inner stylet.

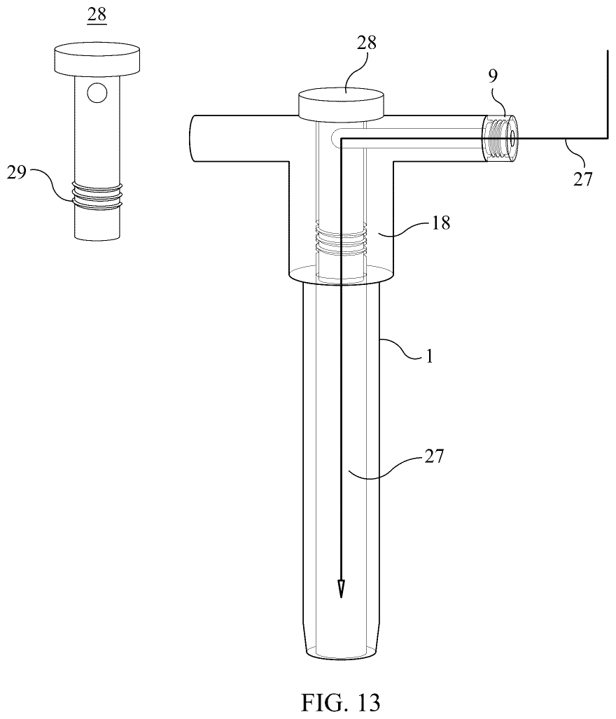

FIGS. 11-13 depict in particular, examples of a first option in which cement (27) is injected directly through handle (9) of the outer sheath. A plug (26) or (28) may be included at the top of the outer sheath handle (2), to allow cement (27) to flow through the outer sheath, without escaping for example, through a top opening (22) in the outer sheath. FIGS. 12 and 13 depict example plugs (26) and (28) that may be used in accordance with these embodiments, but the plugs are not limited thereto. The example plugs may be configured with screw threads (29) to screw into cannula threads of the outer sheath (6) that may be the same or different threads of the outer sheath that are configured for corresponding to threads (12) of the inner stylet (8) at an earlier step in the present processes. These examples have a channel through the handle and main body of the outer sheath for the cement (27) to flow into the tube of the outer sheath and into the vertebrae of a patient, for example during Kyphoplasty methods. FIG. 13 depicts a non-limiting embodiment showing a plug within a cannula instrument or device of the present systems that allow for cement injection directly through the handle of the instrument or device.

FIG. 13 shows an example plug (28) inserted into an opening (22) of the outer sheath (1) and attached to the outer sheath, for example by screwing screw threads (29) of the plug (28) into the outer sheath so the plug does not pop out when cement is inserted into the outer sheath (1).

The plug plugs the location where the inner stylet had previously been inserted, to prevent cement from escaping out that opening at the top of the outer sheath. As shown in FIG. 10, cement may be inserted into a handle of the outer sheath and flows through a channel in the plug and through the outer sheath to the vertebrae of a patient.

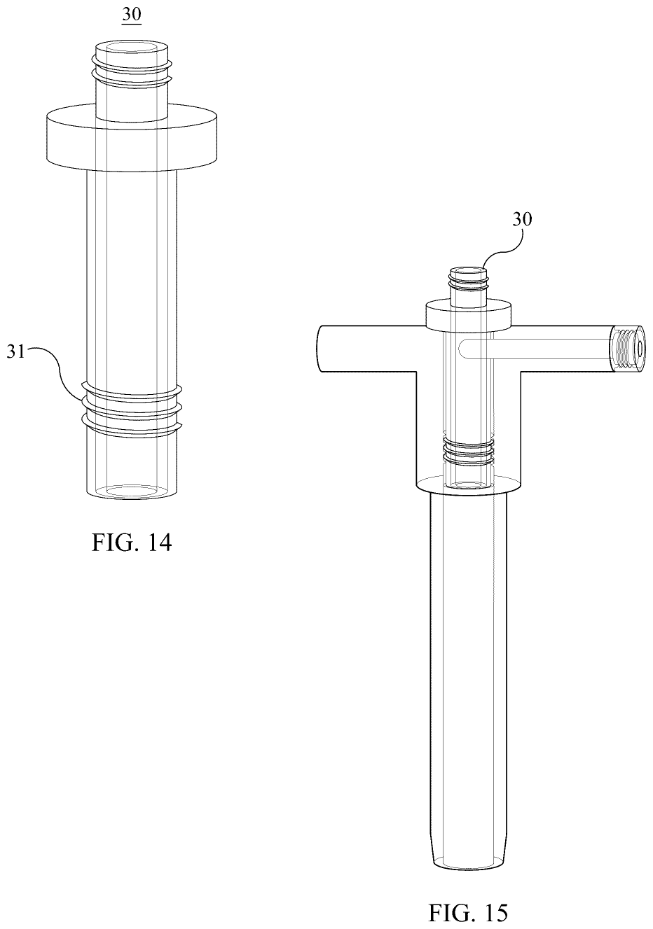

FIGS. 14-15 depict other examples of a possible plug (30) for cement injection, and the plug (30) in use within an example outer sheath (1). According to example embodiments, the plug (30) may have screw threads (31) that screw into the cannula/outer sheath. FIG. 14 depicts a non-limiting embodiment showing a plug (30) configured for insertion within a cannula instrument or device of the present systems that allow for cement injection using small pushers and prefilled cement cannulas inserted into the instrument or device.

FIG. 15 depicts an example plug (3) within an example cannula instrument or device.

Example plugs may have a luer lock that cement can connect to. According to example embodiments, the plug has a hollow option so cement can be injected directly through the cannula. Alternatively, the present plugs may be used with a system including standard pusher tubes that are preloaded with cement passed through.

According to these embodiments, the example plugs may screw into the cannula as discussed above. Then, tubing and/or a direct cement injector is connected to a Luer lock. Optionally, pre-loaded pushers can also be used to push cement down the tube directly or via standard tube with a pusher.

Plugs used in accordance with the present application may be included in various systems of the present application, and/or may be included as part of a kit that includes one or more devices of the present system.

The present application also includes systems that include one or more of the present devices or components thereof. For example, the present systems may include an outer sheath, an inner stylet, and/or a plug, or multiple configurations of any of the above. Example systems may further include a key, a pre-loaded pusher, or cement to be used with the present systems.

Also included are kits that include one or more of the devices or systems or of the present application, or components thereof (assembled or unassembled), and optionally one or more further component(s) related to use of the present devices and systems. In non-limiting examples, kits may include one or more outer sheaths and/or inner stylets that may be of various sizes or configurations. For example, a kit may include multiple inner stylets having different styles or patterns or threading at the tip to allow for different types of biopsies, each inner stylet being configured to fit within an outer sheath. Example kits may also include multiple different types of plugs for insertion into a top hole after removal of the inner stylet.

By way of non-limiting example, systems or kits may optionally include instructions for use, a storage container, a key to start the threading process to advance the outer sheath over the inner stylet, one or more plugs for cement injection, one or more plugs for insertion into a top hole after removal of the inner stylet, cement to be inserted into the patient, and/or other instruments or devices that may be used with the present devices as part of a Kyphoplasty system, including for example visualization components such as x-ray devices and the like. Kits may also include collection containers for collection and/or storage of any biopsy collected from a patient during use of the present devices or systems.

It should be noted that the present devices and systems may be prepared and sold in component parts, or preassembled at least in part. Each of the component parts, and assembled instruments/devices, and kits including one or more parts or assembled devices is within the scope of the present application.

Example methods of the present application include methods of assembling a kyphoplasty device in accordance with the present application. Non-limiting methods include the following: inserting an inner stylet having an inner stylet handle into an opening of an outer sheath having an outer sheath handle; and locking the inner stylet to the outer sheath, to form an assembled kyphoplasty instrument. The present methods may further include screwing the inner stylet into the outer sheath.

Also included herein are methods of performing Kyphoplasty procedures that include the use of one or more of the present devices and/or systems as part of the procedure. Kyphoplasty procedures include multiple steps before and after the portion of the procedure that includes use of the present devices (such as e.g., preparing the surgical site, delivering anesthesia (such as a local anesthetic), implementing visualization of the procedure, closing and dressing the surgical site, etc.). Therefore, the present methods should not be considered to encompass the entire procedure, but rather a portion thereof.

After the present devices are placed within the bone, there are multiple options to obtain a biopsy and/or inject cement without manipulating the handle or passing other instruments within the pedicle. When the present procedures are done under local anesthetic, the less movement there is in and out of the patient spine the better. The present devices, systems and methods provide for less movement in and out of the patient's spine and are therefore superior to previous methods of performing kyphoplasty procedures and previous devices for performing such procedures.

Non-limiting methods of performing kyphoplasty using the present devices and systems in accordance with the present application include the following: inserting an inner stylet having a handle into an opening of an outer sheath having a handle; locking the inner stylet to the outer sheath, to form an assembled kyphoplasty instrument; advancing the assembled kyphoplasty instrument into a desired location of a patient; advancing the outer sheath over the inner stylet to position the outer sheath into a desired position within the patient for cement injection; removing the inner stylet from the outer sheath; plugging the opening of the outer sheath using a plug; and inserting cement through outer sheath into the vertebrae of the patient.

Example methods of the present application may further include removing the outer sheath (including the plug and any cement remaining in the outer sheath) from the patient.

Example methods may further include obtaining a biopsy material from the patient using a tip of the inner stylet, and collecting the biopsy material after removing the inner stylet from the outer sheath.

According to further embodiments, the upper sheath handle has a handle opening that is contiguous with the through-opening. According to examples, inserting cement through the outer sheath may include inserting cement through the handle opening and through the through-opening to a desired location of the patient. Other methods of inserting cement through the outer sheath are contemplated as discussed above.

Also included are methods that include inserting an outer sheath having an outer sheath handle and having a through-opening extending therethrough into a desired position of a patient within the patient for cement injection; inserting an inner stylet having a inner stylet handle into the top opening of the outer sheath; removing the inner stylet from the outer sheath; plugging the top opening of the outer sheath using a plug; and inserting cement through the outer sheath into the vertebrae of the patient. As with other methods discussed above, the methods may further include removing the outer sheath from the patient.

According to non-limiting examples, as with other methods herein, these methods may also include obtaining a biopsy material, for example using the inner stylet, and collecting the biopsy material after removing the inner stylet from the outer sheath.

Cement may be inserted through the outer sheath as discussed herein.

Each component of the present methods may be the same as described herein with respect to the devices, systems and kits of the present application.

According with example embodiments of the present application, the present methods may include locking the inner stylet and outer sheath together using a luer lock, as discussed above.

According with example embodiments of the present application, the present methods may include advancing the outer sheath over the inner stylet by rotating the outer sheath over the inner stylet.

According with example embodiments of the present application, the outer sheath is rotated over the stylet using a key inserted into the handle of the outer sheath or into a luer lock.

According to example embodiments, example methods may also include collecting biopsy samples from the patient, when the inner stylet is removed from the outer sheath. The biopsy samples may include bone pieces removed from the patient when a self tapping tip of the inner stylet is screwed into a patient, or a core biopsy removed from an inner stylet configured for obtaining a core biopsy.

In non-limiting examples, as discussed above, methods may include inserting the cement into the outer sheath through the handle of the outer sheath. Alternatively, the cement may be inserted into the cannula by injecting cement directly through the cannula or by using a standard pusher tube preloaded with cement.

According to non-limiting example embodiments, the present application may include inserting the inner stylet into the outer sheath partially or fully to form a complete cannula instrument, and thereafter inserting the entire instrument into a patient. Alternatively, the outer sheath may be inserted into the patient and thereafter the inner stylet may be inserted into the outer sheath to complete formation of the device within the patient.

According to example embodiments, the present devices and systems will have sterile and disposable packaging.

According to the present application, the patients or subjects are humans, but it is contemplated that the present devices and methods may be used with other mammals.

While the present disclosure has been described in terms of exemplary aspects, those skilled in the art will recognize that the present disclosure can be practiced with modifications that are within the spirit and scope of the present application. The examples provided herein are merely illustrative and are not meant to be an exhaustive list of all possible designs, aspects, applications or modifications of the present disclosure. Accordingly, it is intended that such changes and modifications fall within the scope of the present disclosure.

I claim:

1. A kyphoplasty cannula device system comprising
an outer sheath comprising a main body defining a through-opening extending therethrough, and an outer sheath handle above the main body, the outer sheath handle having a handle opening through at least one end thereof, said outer sheath handle having a top opening that is contiguous with the through-opening of the main body; and
an inner stylet configured to be insertable into the top opening of the outer sheath handle and into the through-opening of the main body.

2. The kyphoplasty cannula device system of claim 1, further comprising
a plug configured to be insertable into the top opening of the outer sheath handle after removal of the inner stylet from the outer sheath.

3. The kyphoplasty cannula device system of claim 1, wherein an inside of the main body defining through-opening includes internal outer sheath screw threads, and the outside of the inner stylet includes inner stylet screw threads configured to correspond to the outer sheath screw threads.

4. The kyphoplasty cannula device system of claim 1, wherein an end of the outer sheath main body distal to the outer sheath handle comprises sharp edges.

5. The kyphoplasty cannula device system of claim 1, wherein a handle of the inner stylet comprises a first lock portion that is configured to lock to a second lock portion of the outer sheath handle.

6. The kyphoplasty cannula device system of claim 1, further comprising an instrument key configured to be insertable into an access port on an end of the outer sheath handle, and configured to engage with the outer sheath handle to rotate the outer sheath over the inner stylet.

7. The kyphoplasty cannula device system of claim 1, wherein the outer sheath handle opening extends to meet up with and be open to the through-opening of the main body, and is configured to allow cement to pass therethrough.

8. The kyphoplasty cannula device system of claim 1, wherein an end of the inner stylet distal to the inner stylet handle comprises a self drilling, self tapping tip.

9. A kit comprising
the kyphoplasty system of claim 1, and
at least one additional component selected from the group consisting of instructions for use, a storage container, an instrument key, a cement plug for cement injection, a plug for insertion into the top opening of the outer sheath after removal of the inner stylet, bone cement, a visualization devise, and an instrument for assembly or use of the kyphoplasty systems.

10. A method comprising
inserting an inner stylet having a inner stylet handle into a top opening of an outer sheath having an outer sheath handle and having a through-opening extending therethrough;
locking the inner stylet to the outer sheath, to form an assembled kyphoplasty instrument;
advancing the assembled kyphoplasty instrument into a desired location of a patient;
advancing the outer sheath over the inner stylet to position the outer sheath into a desired position of a patient within the patient for cement injection;
removing the inner stylet from the outer sheath;
plugging the top opening of the outer sheath using a plug; and
inserting cement through outer sheath into the vertebrae of the patient.

11. The method of claim 10, further comprising removing the outer sheath from the patient.

12. The method of claim 10, further comprising obtaining a biopsy material from the patient using a tip of the inner stylet, and collecting the biopsy material after removing the inner stylet from the outer sheath.

13. The method of claim 10, wherein the upper sheath handle has a handle opening that is contiguous with the through-opening, and wherein inserting cement through the outer sheath comprises inserting cement through the handle opening and through the through-opening to a desired location of the patient.

14. A method comprising
inserting an outer sheath having an outer sheath handle and having a through-opening extending therethrough, into a desired position of a patient for cement injection;
inserting an inner stylet having a inner stylet handle into the top opening of the outer sheath;
removing the inner stylet from the outer sheath;
plugging the top opening of the outer sheath using a plug; and
inserting cement through outer sheath into the vertebrae of the patient.

15. The method of claim 14, further comprising removing the outer sheath from the patient.

16. The method of claim 14, further comprising obtaining a biopsy material from the patient using the inner stylet; and collecting the biopsy material after removing the inner stylet from the outer sheath.

17. The method of claim 14, wherein the upper sheath handle has a handle opening that is contiguous with the through-opening, and wherein inserting cement through the outer sheath comprises inserting cement through the handle opening and through the through-opening to a desired location of the patient.
